(19) United States
(12) Patent Application Publication
(10) Pub. No.: US 2022/0053776 A1
(43) Pub. Date: Feb. 24, 2022

(54) Fulvic Acid Sterilization Deodorant As Well As Preparation Method and Application Thereof

(71) Applicant: Shaanxi University of Science & Technology, Xi'an (CN)

(72) Inventors: Yuhua Niu, Xi'an (CN); Xiaobin Zhu, Shenmu County (CN); Changhui Zhang, Xianyang City (CN); Jie Song, Xi'an (CN); Xingxing Han, Xi'an (CN); Yufang Dou, Tianshui (CN); Yangyang Wang, Yulin City (CN); Xuan Zhao, Xi'an city (CN)

(21) Appl. No.: 17/357,058

(22) Filed: Jun. 24, 2021

(30) Foreign Application Priority Data
Aug. 19, 2020 (CN) 2020108384236

(51) Int. Cl. A01N 65/20; A61L 9/14; A01N 25/30; A01N 65/24; A01N 65/48; A01N 25/04; A01N 65/22

(52) U.S. Cl. CPC A01N 65/20; A61L 9/14; A01N 25/30; A61L 2209/132; A01N 65/48; A01N 25/04; A01N 65/22; A01N 65/24

(57) ABSTRACT

The invention discloses a fulvic acid sterilization deodorant as well as a preparation method and application thereof, and belongs to the technical field of deodorants. Natural polymer compound fulvic acid is modified to prepare a fulvic acid surfactant which is compounded with plant extract to prepare the deodorant with good sterilization and deodorization effects. The fulvic acid with strong hydrophilicity is enhanced in surface activity by introducing a lipophilic end through reaction with a cationic surfactant; an emulsifier is introduced to the fulvic acid solution and the plant extract with strong volatility to prolong interaction between deodorant and odor molecules, achieving an efficient removal purpose. The preparation method is simple in process and low in cost, adopts easily-available raw materials, and can achieve large-scale production. The sterilization deodorant prepared has good deodorization and sterilization performances and can be widely applied to the aspects of odor removal, environmental governance, etc.

### CROSS REFERENCE TO RELATED APPLICATION

The invention belongs to the technical field of deodorants, and specifically relates to a fulvic acid sterilization deodorant as well as a preparation method and application thereof.

### BACKGROUND OF THE INVENTION

With greater attention to environmental protection and rising environmental awareness of the people, it is becoming increasingly urgent to treat the odor generated by domestic garbage, farms, and industrial production. The generated odor not only pollutes the air quality in residential areas, but also causes the spread of diseases, seriously hindering the development of people's physical and mental health.

At present, spraying deodorants into the air is a common treatment method, but the addition of organic solvents to traditional deodorants will cause secondary pollution and hidden danger to human health although the deodorization effect can be achieved.

### SUMMARY OF THE INVENTION

To solve the problems, the invention discloses a fulvic acid sterilization deodorant as well as a preparation method and application thereof. The preparation method is simple in process and low in cost, adopts easily-available raw materials, and can achieve large-scale production. The sterilization deodorant prepared has good deodorization and sterilization performances.

The invention adopts the following technical scheme:

The invention discloses a fulvic acid sterilization deodorant, including the following raw materials in parts by mass:

8-12 parts of a fulvic acid surfactant, 20-40 parts of gleditsia sinensis extract, 10-30 parts of camphor extract, 3-6 parts of ginger extract, 20-44 parts of peppermint extract, 0.1-2 parts of an emulsifier, 1-3 parts of a penetrating agent and 40-60 parts of deionized water.

The invention discloses a preparation method for the fulvic acid sterilization deodorant, in parts by mass, including the following steps:

Step 1: taking and completely dissolving 8-12 parts of fulvic acid surfactant into 40-60 parts of deionized water by stirring in a 50° C. water bath to obtain a uniform solution A;

Step 2: adding 0.1-2 parts of the emulsifier into the solution A, and continuously emulsifying for 30 mi at 50° C. to obtain pre-emulsion B;

Step 3: mixing 20-40 parts of gleditsia sinensis extract, 10-30 parts of camphor extract, 3-6 parts of ginger extract and 20-44 parts of peppermint extract to obtain a mixed solution, and ultrasonically treating the mixed solution for 25-30 min at the normal temperature to obtain a solution C;

Step 4: adding the solution C and 1-3 parts of the penetrating agent into the pre-emulsion B, and uniformly stirring at the room temperature to obtain the fulvic acid sterilization deodorant.

Preferably, the preparation method for the fulvic acid sterilization deodorant includes the following steps: reacting fulvic acid and the cationic surfactant for 3-4 h in a 45-60° C. water bath in a mass ratio of 42 to 1, drying the filtrate at 60° C. after vacuum filtration to prepare the fulvic acid sterilization deodorant.

Further preferably, the cationic surfactant is dodecyl trimethylammonium chloride, cetyltrimethylammonium chloride or octadecyl trimethylammonium chloride.

Preferably, the preparation method for the gleditsia sinensis extract includes the following steps: cleaning and adding gleditsia sinensis into equal volume of deionized water to soak for 4 h, squeezing and adopting a 100-mesh screen to filter, and concentrating the filtrate to 1.3 g/cm³, thereby obtaining the gleditsia sinensis extract.

Preferably, the preparation method for the camphor extract includes the following steps: crushing camphor to pass through a 80-mesh screen, and mixing camphor powder and petroleum ether in a solid-liquid ratio of 1 to 20 to obtain a mixture, and ultrasonically extracting the mixture for 50 min at 70° C. to obtain the camphor extract.

Preferably, the preparation method for the ginger extract includes the following steps: crushing gingers to pass through a 80-mesh screen, mixing the ginger powder with absolute ethyl alcohol in a solid-liquid ratio of 1 to 20 to obtain a mixture, and ultrasonically extracting the mixture for 50 min at 70° C. to obtain the ginger extract.

Preferably, the preparation method for peppermint extract includes the following steps: crushing peppermints to pass through a 80-mesh screen, mixing peppermint powder with petroleum ether in a solid-liquid ratio of 1 to 12 to soak at 40° C. to obtain the pepper peppermint extract.

Preferably, the emulsifier is Tween-20 and the penetrating agent is fatty alcohol polyoxyethylene ether.

The invention discloses application of the fulvic acid sterilization deodorant prepared by the preparation method as a sterilization deodorant. After being diluted with deionized water, the fulvic acid sterilization deodorant is sprayed into air in the form of mist spray through ultrasonic atomizing equipment.

Compared with the prior art, the invention has the following beneficial technical effects:

The fulvic acid sterilization deodorant disclosed by the invention is a natural polymer compound, and its structural unit contains multiple functional groups such as phenolic hydroxyl group, alcoholic hydroxyl group, carboxyl group, enol group, sulfonic acid group, etc. In terms of structure, it contains both non-polar lipophilic groups and polar hydrophilic group, is more environmentally friendly, non-toxic, and good in environmental compatibility compared with chemically synthesized surfactants, has better selectivity and specificity as well as more active group at an extreme temperature, an extreme pH value and extreme salt concentration, and has two-way effects of adjusting acidity and alkalinity. It is compounded with a variety of plant extracts, most of effective molecules in plant extracts of gleditsia sinensis extract, camphor extract, ginger and peppermint contain active groups such as conjugated double bonds, which can interact with odor molecules, and cooperate with a variety of raw materials to achieve good deodorization and sterilization performance. At the same time, because the fulvic acid solution and the plant extract have strong volatility, in order to prolong the interaction between the deodorant molecules and the odor molecules, the emulsifier is introduced to achieve the purpose of efficient removal. Due to the poor penetration performance of spraying the deodorant on the surface of the target, the penetration is improved by adding the penetrating agent to promote the deodorant molecules act on the target better, thus improving the deodorization efficiency. The raw materials of the sterilization deodorant are safe, environmentally friendly, reliable, and easy to degrade.

According to the preparation method for the fulvic acid sterilization deodorant disclosed by the invention, a fulvic acid surfactant is compounded with the plant extract to prepare the deodorant with good deodorization and sterilization effects. The method adopts cheap and easily-available raw materials, is simple in operation and low in cost, and has good economic and social benefits.

Further, the fulvic acid surfactant is prepared by compounding fulvic acid with the cationic surfactant. The fulvic acid with strong hydrophilicity is enhanced in surface activity by introducing a lipophilic end through reaction with the cationic surfactant.

Further, the cationic surfactant adopts dodecyl trimethyl ammonium chloride, cetyltrimethyl ammonium chloride or octadecyl trimethyl ammonium chloride; lipophilic groups with different carbon chain lengths can be introduced onto fulvic acid molecules, so that a lipophilic-hydrophilic balance value of the fulvic acid is improved to obtain fulvic acid surfactants with different surface activities.

Further, the ultrasonic method is used to extract 4 plant liquids, which is simple to operate, short in time and high in efficiency.

Further, the emulsifier adopts Tween-20, which has a good emulsifying effect on vegetable oils, and is commonly used for preparation of sterile and sterilizing agents. Tween-20 is used to emulsify the water-phase aqueous fulvic acid surfactant and the oil-phase plant extract, and also improves the sterilization performance The fatty alcohol polyoxyethylene ether used in the penetrating agent is a non-ionic surfactant, which has good decontamination and cleaning capabilities, can reduce the surface tension of the sterilization deodorant, and improves performance of the fulvic acid sterilization deodorant through its good compatibility and synergistic effect with the emulsifier Tween-20.

According to the application of the fulvic acid sterilization deodorant prepared by the preparation method of the fulvic acid sterilization deodorant disclosed by the invention in the field of odor removal and environmental governance. After being diluted with deionized water by different multiples, the fulvic acid sterilization deodorant is sprayed into the air in the form of mist spray through the ultrasonic atomizing equipment. Through the unique structure of the fulvic acid surfactant, odor molecules of hydrogen sulfide, ammonia, mercaptans, organic sulfur, organic amines and some inorganic substance in odor can be degraded and converted into non-toxic and harmless substances. At the same time, the plant extract contains a large number of active groups, which can achieve the purpose of removing odors through acid-base reactions, catalytic oxidation reactions, and redox reactions.

### DETAILED DESCRIPTION OF THE PREFERRED EMBODIMENTS

The invention will be further described in detail below in conjunction with specific embodiments, which are for explanation rather than limitation of the invention. The parts involved in the embodiment are all calculated in parts by mass.

### Embodiment 1

Fulvic acid and dodecyl trimethyl ammonium chloride reacted for 3 h in a 45° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

8 parts of fulvic acid surfactant was taken and completely dissolved into 40 parts of deionized water by stirring in a 50° C. water bath to obtain a uniform solution A; 0.1 part of the emulsifier was added into the solution A, and continuously emulsified for 30 min at 50° C. to obtain pre-emulsion B; 20 parts of gleditsia sinensis extract, 10 parts of camphor extract, 3 parts of ginger extract and 20 parts of peppermint extract were taken and mixed to obtain a mixed solution, and the mixed solution was ultrasonically treated for 25 min at the normal temperature to obtain a solution C; 1 part of fatty alcohol-polyoxyethylene ether and the solution C were added into the pre-emulsion B, and were uniformly stirred at the room temperature to obtain the fulvic acid sterilization deodorant.

### Embodiment 2

Fulvic acid and dodecyl trimethyl ammonium chloride reacted for 3.5 h in a 50° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

10 parts of fulvic acid surfactant was taken and completely dissolved into 50 parts of deionized water by stirring in a 50° C. water bath to obtain a uniform solution A; 1 part of the Tween-20 was added into the solution A, and continuously emulsified for 30 min at 50° C. to obtain pre-emulsion B; 30 parts of gleditsia sinensis extract, 20 parts of camphor extract, 4 parts of ginger extract and 32 parts of peppermint extract were taken and mixed to obtain a mixed solution, and the mixed solution was ultrasonically treated for 30 min at the normal temperature to obtain a solution C; 2 parts of fatty alcohol-polyoxyethylene ether and the solution C were added into the pre-emulsion B, and were uniformly stirred at the room temperature to obtain the fulvic acid sterilization deodorant.

### Embodiment 3

Fulvic acid and dodecyl trimethyl ammonium chloride reacted for 4 h in a 60° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

12 parts of fulvic acid surfactant was taken and completely dissolved into 60 parts of deionized water by stirring in a 50° C. water bath to obtain a uniform solution A; 2 part of the Tween-20 was added into the solution A, and continuously emulsified for 30 min at 50° C. to obtain pre-emulsion B; 40 parts of gleditsia sinensis extract, 30 parts of camphor extract, 6 parts of ginger extract and 44 parts of peppermint extract were taken and mixed to obtain a mixed solution, and the mixed solution was ultrasonically treated for 35 min at the normal temperature to obtain a solution C; 3 parts of fatty alcohol-polyoxyethylene ether and the solution C were added into the pre-emulsion B, and were uniformly stirred at the room temperature to obtain the fulvic acid sterilization deodorant.

### Embodiment 4

Fulvic acid and dodecyl trimethyl ammonium chloride reacted for 3 h in a 45° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

8 parts of fulvic acid surfactant was taken and completely dissolved into 40 parts of deionized water by stirring in a 50° C. water bath to obtain a uniform solution A; 0.1 part of the Tween-20 was added into the solution A, and continuously emulsified for 30 min at 50° C. to obtain pre-emulsion B; 20 parts of gleditsia sinensis extract, 10 parts of camphor extract, 3 parts of ginger extract and 20 parts of peppermint extract were taken and mixed to obtain a mixed solution, and the mixed solution was ultrasonically treated for 25 min at the normal temperature to obtain a solution C; 1 part of fatty alcohol-polyoxyethylene ether and the solution C were added into the pre-emulsion B, and were uniformly stirred at the room temperature to obtain the fulvic acid sterilization deodorant.

### Embodiment 5

Fulvic acid and dodecyl trimethyl ammonium chloride reacted for 3.5 h in a 50° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

10 parts of fulvic acid surfactant was taken and completely dissolved into 50 parts of deionized water by stirring in a 50° C. water bath to obtain a uniform solution A; 1 part of the Tween-20 was added into the solution A, and continuously emulsified for 30 minat 50° C. to obtain pre-emulsion B; 30 parts of gleditsia sinensis extract, 20 parts of camphor extract, 4 parts of ginger extract and 32 parts of peppermint extract were taken and mixed to obtain a mixed solution, and the mixed solution was ultrasonically treated for 30 min at the normal temperature to obtain a solution C; 2 parts of fatty alcohol-polyoxyethylene ether and the solution C were added into the pre-emulsion B, and were uniformly stirred at the room temperature to obtain the fulvic acid sterilization deodorant.

### Embodiment 6

Fulvic acid and dodecyl trimethyl ammonium chloride reacted for 4 h in a 60° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

12 parts of fulvic acid surfactant was taken and completely dissolved into 60 parts of deionized water by stirring in a 50° C. water bath to obtain a uniform solution A; 2 parts of the Tween-20 were added into the solution A, and continuously emulsified for 30 min at 50° C. to obtain pre-emulsion B; 40 parts of gleditsia sinensis extract, 30 parts of camphor extract, 6 parts of ginger extract and 44 parts of peppermint extract were taken and mixed to obtain a mixed solution, and the mixed solution was ultrasonically treated for 35 min at the normal temperature to obtain a solution C; 3 parts of fatty alcohol-polyoxyethylene ether and the solution C were added into the pre-emulsion B, and were uniformly stirred at the room temperature to obtain the fulvic acid sterilization deodorant.

### Embodiment 7

Fulvic acid and dodecyl trimethyl ammonium chloride reacted for 3 h in a 45° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

8 parts of fulvic acid surfactant was taken and completely dissolved into 40 parts of deionized water by stirring in a 50° C. water bath to obtain a uniform solution A; 0.1 part of the Tween-20 was added into the solution A, and continuously emulsified for 30 min at 50° C. to obtain pre-emulsion B; 20 parts of gleditsia sinensis extract, 10 parts of camphor extract, 3 parts of ginger extract and 20 parts of peppermint extract were taken and mixed to obtain a mixed solution, and the mixed solution was ultrasonically treated for 25 min at the normal temperature to obtain a solution C; and 1 part of fatty alcohol-polyoxyethylene ether and the solution C were added into the pre-emulsion B, and were uniformly stirred at the room temperature to obtain the fulvic acid sterilization deodorant.

### Embodiment 8

Fulvic acid and dodecyl trimethyl ammonium chloride reacted for 3.5 h in a 50° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

10 parts of fulvic acid surfactant was taken and completely dissolved into 50 parts of deionized water by stirring in a 50° C. water bath to obtain a uniform solution A; 1 part of the Tween-20 was added into the solution A, and continuously emulsified for 30 min at 50° C. to obtain pre-emulsion B; 30 parts of gleditsia sinensis extract, 20 parts of camphor extract, 4 parts of ginger extract and 32 parts of peppermint extract were taken and mixed to obtain a mixed solution, and the mixed solution was ultrasonically treated for 30 min at the normal temperature to obtain a solution C; 2 parts of fatty alcohol-polyoxyethylene ether and the solution C were added into the pre-emulsion B, and were uniformly stirred at the room temperature to obtain the fulvic acid sterilization deodorant.

### Embodiment 9

Fulvic acid and dodecyl trimethyl ammonium chloride reacted for 4 h in a 60° C. water bath in a mass ratio of 42 to 1, the filtrate was dried after vacuum filtration to obtain a fulvic acid surfactant; gleditsia sinensis was cleaned and added into equal volume of deionized water to soak for 4 h, squeezed and filtered with a 100-mesh screen, and the filtrate was concentrated to 1.3 g/cm³ to obtain the gleditsia sinensis extract; camphor was crushed to pass through a 80-mesh screen, camphor powder was mixed with petroleum ether in a solid-liquid ratio of 1 to 20 to obtain a mixture, and the mixture was ultrasonically treated for 50 min at 70° C. to obtain the camphor extract; gingers were crushed to pass through a 80-mesh screen, the ginger powder was mixed with absolute ethyl alcohol in a solid-liquid ratio of 1 to 20 to obtain a mixture, and the mixture was ultrasonically extracted for 50 min at 70° C. to obtain the ginger extract; and peppermints were crushed to pass through a 80-mesh screen, peppermint powder was mixed with petroleum ether in a solid-liquid ratio of 1 to 12 to soak at 40° C. to obtain the pepper peppermint extract.

12 parts of fulvic acid surfactant was taken and completely dissolved into 60 parts of deionized water by stirring in a 50° C. water bath to obtain a uniform solution A; 2 parts of the Tween-20 were added into the solution A, and continuously emulsified for 30 min at 50° C. to obtain pre-emulsion B; 40 parts of gleditsia sinensis extract, 30 parts of camphor extract, 6 parts of ginger extract and 44 parts of peppermint extract were taken and mixed to obtain a mixed solution, and the mixed solution was ultrasonically treated for 35 min at the normal temperature to obtain a solution C; and 3 parts of fatty alcohol-polyoxyethylene ether and the solution C were added into the pre-emulsion B, and were uniformly stirred at the room temperature to obtain the fulvic acid sterilization deodorant.

The preferred embodiments of the invention have been described in detail above, but the invention is not limited to the above-mentioned embodiments. Within the scope of knowledge possessed by those of ordinary skill in the art, various changes can be made without departing from the purpose of the invention. There is no need to and cannot give an exhaustive list of all implementation methods. The obvious changes or modifications derived from this are still within the protection scope of the invention.

What is claimed is:

1. A fulvic acid sterilization deodorant, comprising the following raw materials in parts by mass: 8-12 parts of a fulvic acid surfactant, 20-40 parts of gleditsia sinensis extract, 10-30 parts of camphor extract, 3-6 parts of ginger extract, 20-44 parts of peppermint extract, 0.1-2 parts of an emulsifier, 1-3 parts of a penetrating agent and 40-60 parts of deionized water.

2. A preparation method for the fulvic acid sterilization deodorant, in parts by mass, comprising the following steps:
step 1: taking and completely dissolving 8-12 parts of fulvic acid surfactant into 40-60 parts of deionized water by stirring in a 50° C. water bath to obtain a uniform solution A;
step 2: adding 0.1-2 parts of the emulsifier into the solution A, and continuously emulsifying for 30 min at 50° C. to obtain pre-emulsion B;
step 3: mixing 20-40 parts of gleditsia sinensis extract, 10-30 parts of camphor extract, 3-6 parts of ginger extract and 20-44 parts of peppermint extract to obtain a mixed solution, and ultrasonically treating the mixed solution for 25-30 min at the normal temperature to obtain a solution C;
step 4: adding the solution C and 1-3 parts of the penetrating agent into the pre-emulsion B, and uniformly stirring at the room temperature to obtain the fulvic acid sterilization deodorant.

3. The preparation method for the fulvic acid sterilization deodorant according to claim 2, wherein preparation method for the fulvic acid sterilization deodorant comprises the following steps: reacting fulvic acid and the cationic surfactant for 3-4 h in a 45-60° C. water bath in a mass ratio of 42 to 1, drying the filtrate at 60° C. after vacuum filtration to prepare the fulvic acid sterilization deodorant.

4. The preparation method for the fulvic acid sterilization deodorant according to claim 3, wherein the cationic surfactant is dodecyl trimethylammonium chloride, cetyltrimethylammonium chloride or octadecyl trimethylammonium chloride.

5. The preparation method for the fulvic acid sterilization deodorant according to claim 2, wherein the preparation method for the gleditsia sinensis extract comprises the following steps: cleaning and adding gleditsia sinensis into equal volume of deionized water to soak for 4 h, squeezing and adopting a 100-mesh screen to filter, and concentrating the filtrate to 1.3 g/cm³, thereby obtaining the gleditsia sinensis extract.

6. The preparation method for the fulvic acid sterilization deodorant according to claim 2, wherein the preparation method for the camphor extract comprises the following steps: crushing camphor to pass through a 80-mesh screen, and mixing camphor powder and petroleum ether in a solid-liquid ratio of 1 to 20 to obtain a mixture, and ultrasonically extracting the mixture for 50 min at 70° C. to obtain the camphor extract.

7. The preparation method for the fulvic acid sterilization deodorant according to claim 2, wherein the preparation method for the ginger extract comprises the following steps: crushing gingers to pass through a 80-mesh screen, mixing the ginger powder with absolute ethyl alcohol in a solid-liquid ratio of 1 to 20 to obtain a mixture, and ultrasonically extracting the mixture for 50 min at 70° C. to obtain the ginger extract.

8. The preparation method for the fulvic acid sterilization deodorant according to claim 2, wherein the preparation method for peppermint extract comprises the following steps: crushing peppermints to pass through a 80-mesh screen, mixing peppermint powder with petroleum ether in a solid-liquid ratio of 1 to 12 to soak at 40° C. to obtain the pepper peppermint extract.

9. The preparation method for the fulvic acid sterilization deodorant according to claim 2, wherein the emulsifier is Tween-20 and the penetrating agent is fatty alcohol polyoxyethylene ether.
